(19) United States
(12) Patent Application Publication
(10) Pub. No.: US 2025/0222075 A1
(43) Pub. Date: Jul. 10, 2025

(54) NOVEL APPLICATION OF HUMAN AIRWAY SURFACTANT, BPIFA1, AS PROPHYLACTIC ANTIVIRAL AGAINST RESPIRATORY SYNCYTIAL VIRUS (RSV)

(71) Applicants: Sanford Burnham Prebys Medical Discovery Institute, La Jolla, CA (US); The Regents of the University of California, Oakland, CA (US)

(72) Inventors: Rachael MCVICAR, La Jolla, CA (US); Evan Y. SNYDER, La Jolla, CA (US); Sandra LEIBEL, San Marcos, CA (US)

(21) Appl. No.: 19/015,071

(22) Filed: Jan. 9, 2025

Related U.S. Application Data
(60) Provisional application No. 63/619,215, filed on Jan. 9, 2024.

(51) Int. Cl. A61K 38/17; A61P 31/14

(52) U.S. Cl. CPC A61K 38/395; A61P 31/14

(57) ABSTRACT

Provided herein are methods and compositions suitable for treating or preventing a respiratory illness and/or condition.

### CROSS REFERENCE

This application claims the benefit of U.S. Patent Application No. 63/619,215, filed Jan. 9, 2024.

### BACKGROUND

Respiratory infections and conditions such as those caused by respiratory syncytial virus (RSV) is the most common infection in infants. Further RSV is the 2^{nd} leading cause of death globally in children under 5 years old. There is a need for antivirals, prophylactics, and/or treatments that can be used against respiratory infections and conditions, especially in children.

### SUMMARY

Described herein are methods for treating a subject prophylactically for a respiratory disease or condition with a therapeutically effective amount of a biologically active protein.

Aspects disclosed herein provide methods for preventing or treating a respiratory infection in a human subject comprising: administering to the human subject a therapeutically effective amount of BPI fold containing family A member 1 (BPIFA1) protein, wherein the respiratory infection is caused by a respiratory virus. In some embodiments, the respiratory virus comprises adenovirus, coronavirus, parainfluenza, parvovirus, respiratory syncytial virus (RSV), or any combination thereof. In some embodiments, the respiratory virus is RSV. In some embodiments, the BPIFA1 protein is administered during the latent phase. In some embodiments, the BPIFA1 protein is administered during the incubation phase. In some embodiments, the BPIFA1 protein is administered following known exposure to a respiratory virus. In some embodiments, the BPIFA1 protein is administered prophylactically. In some embodiments, the BPIFA1 protein is administered following suspected exposure to a respiratory virus. In some embodiments, the subject is susceptible to the respiratory infection. In some embodiments, the subject is at risk of contracting the respiratory infection. In some embodiments, the subject is at risk for complications from the respiratory infection. In some embodiments, wherein the human subject is male, female or intersex according to American Medical Associations' definitions. In some embodiments, the human subject is a neonate, infant, child, adolescent, adult or senior according to American Medical Associations' definitions. In some embodiments, the human subject is a neonate, infant, or child according to American Medical Associations' definitions. In some embodiments, the infant is premature. In some embodiments, the human subject is a senior according to American Medical Associations' definitions. In some embodiments, the human has a preexisting condition. In some embodiments, the preexisting condition comprises a heart, lung, neuromuscular condition, immune condition, or any combination thereof. In some embodiments, the preexisting condition comprises a lung, neuromuscular condition, immune condition, or any combination thereof. In some embodiments, the heart condition comprises a congenital heart condition. In some embodiments, the lung condition comprises a chronic lung condition. In some embodiments, the immune condition comprises being immunocompromised. In some embodiments, the lung condition comprises cystic fibrosis. In some embodiments, the BPIFA1 protein comprises a human BPIFA1 protein. In some embodiments, the BPIFA1 protein has 95% amino acid identity to human BPIFA1 protein. In some embodiments, the BPIFA1 protein has 90% amino acid identity to human BPIFA1 protein. In some embodiments, the BPIFA1 protein has 80% amino acid identity to human BPIFA1 protein. In some embodiments, the BPIFA1 protein has 70% amino acid identity to human BPIFA1 protein. In some embodiments, the BPIFA1 protein comprises residues 40 to 234 of human BPIFA1 protein. In some embodiments, the BPIFA1 protein has 90% amino acid identity residues 40 to 234 of human BPIFA1 protein. In some embodiments, the BPIFA1 protein has 80% amino acid identity residues 40 to 234 of human BPIFA1 protein. In some embodiments, the BPIFA1 protein has 70% amino acid identity residues 40 to 234 of human BPIFA1 protein. In some embodiments, the BPIFA1 protein comprises at least residues 87-92 of human BPIFA1 protein. In some embodiments, the BPIFA1 protein is administered by inhalation. In some embodiments, the BPIFA1 protein is administered through an incubation tube. In some embodiments, the BPIFA1 protein is administered as an aerosol. In some embodiments, the BPIFA1 protein is administered by an infusion. In some embodiments, the BPIFA1 protein is administered by a bolus. In some embodiments, the BPIFA1 protein is administered as a single dose. In some embodiments, the BPIFA1 protein is administered as several doses. In some embodiments, the several doses comprise two doses. In some embodiments, the two doses are administered 24 hours apart. In some embodiments, the two doses are administered 12 hours apart. In some embodiments, the two doses are administered 8 hours apart. In some embodiments, the two doses are administered 6 hours apart. In some embodiments, the two doses are administered 4 hours apart. In some embodiments, the method further comprises administering the BPIFA1 protein in combination with another surfactant. In some embodiments, the another surfactant is an airway surfactant or an alveolar surfactant. In some embodiments, the another surfactant is from porcine, bovine, or synthetic origin. In some embodiments, the another surfactant is selected from any one of the following Survanta®, Infasurf®, Alveofact®, and Curosurf®. In some embodiments, the another surfactant is Curosurf®. In some embodiments, the amount of BPIFA1 protein per dose is 25 mg/kg/dose, 50 mg/kg/dose, 100 mg/kg/dose, 150 mg/kg/dose, 200 mg/kg/dose, 250 mg/kg/dose, 300 mg/kg/dose, 350 mg/kg/dose, 400 mg/kg/dose, 450 mg/kg/dose, or 500 mg/kg/dose of BPIFA1 or an amount of BPIFA1 protein per dose within a range defined by any of the preceding values. In some embodiments, the therapeutically effective amount of BPIFA1 protein is 25 mg/kg/day, 50 mg/kg/day, 100 mg/kg/day, 150 mg/kg/day, 200 mg/kg/day, 250 mg/kg/day, 300 mg/kg/day, 350 mg/kg/day, 400 mg/kg/day, 450 mg/kg/day, 500 mg/kg/day, or 1000 mg/kg/day, or an amount of BPIFA1 protein within a range defined by any of the preceding values.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A shows immunofluorescence imaging for RSV nucleoprotein fluorescence between various conditions.

FIG. 1B shows violin plots of RSV nucleoprotein fluorescence between various conditions calculated with 12 negative biological replicates and 16 positive and test replicates with 4 images per replicate.

FIG. 2 depicts a workflow for analysis of surfactants as antivirals.

FIG. 3A shows immunofluorescence imaging for RSV nucleoprotein fluorescence between various conditions.

FIG. 3B shows bar graphs of nucleoprotein fluorescence between various conditions calculated with 12 biological replicates per treatment.

### DETAILED DESCRIPTION

The present disclosure is based, at least in part, on the surprising discovery that BPIFA1 may be used to treat and/or prevent respiratory viral infections. In particular, the use of BPIFA1 may be used to treat and/or prevent respiratory infections in premature infants.

Disclosed herein are methods and compositions for treating and/or preventing a respiratory infection in a subject. The subject may be a patient. The subject may be administered a biologically active protein, such as for example a surfactant. The surfactant may be a pulmonary surfactant. The surfactant may be a lung surfactant. The surfactant may be an alveolar surfactant. The surfactant may be an airway surfactant. The surfactant may be derived from human, bovine, or porcine. The surfactant may be synthetic. The administration of the biologically active protein (e.g., surfactant) can be used to prevent or treat a respiratory infection in a subject. The surfactant may be administered with another biologically active protein, such as for example an another surfactant. The another surfactant may be a pulmonary surfactant. The another surfactant may be a lung surfactant. The another surfactant may be an alveolar surfactant. The another surfactant may be an airway surfactant. The another surfactant may be derived from human, bovine, or porcine. The another surfactant may be synthetic. The subject may be an infant. The subject may be a newborn. The subject may be a neonate. The subject may have preexisting condition. The subject may be a senior. The subject may be greater than 65 years old. The subject may be at risk for serious complications from a respiratory infection. The respiratory infection may be from a virus, such as for example respiratory syncytial virus (RSV).

### Definitions

The term “pharmaceutically acceptable” it is meant the carrier, diluent or excipient must be compatible with the other ingredients of the formulation.

The term “pharmaceutically acceptable excipient” and “pharmaceutically acceptable carrier” refer to a substance that aids the administration of an active agent to and absorption by a subject and can be included in the compositions of the present disclosure without causing a significant adverse toxicological effect on the patient.

The term “treat”, “treating” and “treatment” and the like refer to any indicia of success to treat, in the treating of, in the treatment of, to ameliorate, in the ameliorating of, in the amelioration of, to prevent, in the preventing of, or in the prevention of an infection, pathology or condition, including any objective or subjective parameter such as abatement; remission; diminishing of symptoms or making the infection, pathology or condition more tolerable to the patient; slowing in the rate of degeneration or decline; making the final point of degeneration less debilitating; improving a patient's physical or mental well-being. The treatment, amelioration, or prevention of symptoms can be based on objective or subjective parameters.

The term “treat”, “treating” and “treatment” and the like can also be used to mean obtaining a desired pharmacological and physiological effect. The effect may be prophylactic in terms of preventing or partially preventing a disease, symptom or condition thereof and/or may be therapeutic in terms of a partial or complete cure of an infection, condition, symptom or adverse effect attributed to the disease. The term “treatment” as used herein covers any treatment of an infection in a mammal, particularly a human, and includes: (a) preventing the infection from occurring in a subject which may be at risk of the infection but has not yet been diagnosed as having it such as a preventive early asymptomatic intervention; (b) inhibiting the infection, i.e., arresting its development; or relieving the infection, i.e., causing regression of the infection and/or its symptoms or conditions such as improvement or remediation of damage.

The term “patient” or “subject” in need thereof refers to a living organism suffering from, prone to a condition, or suspected to have an infection or condition that can be treated by administration of a pharmaceutical composition as provided herein. The term “patient” or “subject” in need thereof refers to a living organism that is prophylactically administration of a pharmaceutical composition as provided herein. The term “subject” as used herein refers to mammals. For examples, mammals contemplated by the present disclosure include human, primates, domesticated animals such as cattle, sheep, pigs, horses, laboratory rodents, other pets and the like.

The term “protein” refers to a recombinant protein, a purified protein, or a synthesized protein.

The term “administering” refers to intratracheal administration, administration as an inhalant, oral administration, administration as a suppository, topical contact, parenteral, intravenous, intraperitoneal, intramuscular, intralesional, intranasal or subcutaneous administration, intrathecal administration, or the implantation of a slow-release device e.g., a mini-osmotic pump, to the subject.

The term “therapeutically effective amount or dose” or “therapeutically sufficient amount or dose” or “effective or sufficient amount or dose” refer to a dose that produces therapeutic effects for which it is administered. The exact dose will depend on the purpose of the treatment.

The singular form “a”, “an”, and “the” include plural references unless the context clearly dictates otherwise. For example, the term “a cell” includes one or more cells, including mixtures thereof. “A and/or B” is used herein to include all of the following alternatives: “A”, “B”, “A or B”, and “A and B.”

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

As used herein, the modifier “about” when referring to a number or a numerical range means that the number or numerical range referred to is an approximation, for example, within experimental variability and/or statistical experimental error, and thus the number or numerical range may vary up to ±10% of the stated number or numerical range.

All ranges disclosed herein also encompass any and all possible sub-ranges and combinations of sub-ranges thereof. Any listed range can be recognized as sufficiently describing and enabling the same range being broken down into at least equal halves, thirds, quarters, fifths, tenths, and so forth. As a non-limiting example, each range discussed herein can be readily broken down into a lower third, middle third and upper third, and the like. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

### Biologically Active Protein

Aspects disclosed herein comprise a biologically active protein. The biologically active protein may be a surfactant. The surfactant may be a pulmonary surfactant. The surfactant may be a lung surfactant. The surfactant may be an alveolar surfactant. The surfactant may be an airway surfactant. The surfactant may be derived from a human surfactant. The surfactant may be derived from a bovine surfactant. The surfactant may be derived from a porcine surfactant. The surfactant may be synthetic. The surfactant may be derived from BPI fold containing family A member 1 (BPIFA1) protein. The BPIFA1 protein may also be known as LUNX, NASG, PLUNC; SPURT, SPLUNC1, or bA49G10.5.

The BPIFA1 protein may be a full-length BPIFA1 protein. The BPIFA1 protein may be a full-length BPIFA1 protein with 100% amino acid identity to variant 1 BPIFA1 protein (NCBI accession: NP_057667.1). The BPIFA1 protein may be a full-length BPIFA1 protein with 100% amino acid identity to variant 2 BPIFA1 protein (NCBI accession: NP_570913.1). The BPIFA1 protein may be a full-length BPIFA1 protein with 100% amino acid identity to variant 3 BPIFA1 protein (NCBI accession: NP_001230122.1). The BPIFA1 protein may be a full-length BPIFA1protein with 100% amino acid identity to isoform 1 BPIFA1 protein (UniProt ID: Q9NP55-1). The BPIFA1 protein may be a full-length BPIFA1 protein with 100% amino acid identity to isoform 2 BPIFA1 protein (UniProt ID: Q9NP55-2). The BPIFA1 protein may be a partial BPIFA1 protein. The partial BPIFA1 protein may be derived from variant 1 BPIFA1 protein (NCBI accession: NP_057667.1). The partial BPIFA1 protein may be derived from variant 2 BPIFA1 protein (NCBI accession: NP_570913.1). The partial BPIFA1 protein may be derived from variant 3 BPIFA1 protein (NCBI accession: NP_001230122.1). The partial BPIFA1 protein may be derived from isoform 1 BPIFA1 protein (UniProt ID: Q9NP55-1). The partial BPIFA1 protein may be derived from isoform 2 BPIFA1 protein (UniProt ID: Q9NP55-2).

The BPIFA1 protein may be a partial BPIFA1 protein. The BPIFA1 protein may have at least 95% amino acid identity to a human BPIFA1 protein, such as for example any one of NP_057667.1, NP_570913.1, NP_001230122.1, Q9NP55-1, or Q9NP55-2. The BPIFA1 protein may have at least 90% amino acid identity to a human BPIFA1 protein, such as for example any one of NP_057667.1, NP_570913.1, NP_001230122.1, Q9NP55-1, or Q9NP55-2. The BPIFA1 protein may have at least 85% amino acid identity to a human BPIFA1 protein, such as for example any one of NP_057667.1, NP_570913.1, NP_001230122.1, Q9NP55-1, or Q9NP55-2. The BPIFA1 protein may have at least 80% amino acid identity to a human BPIFA1 protein, such as for example any one of NP_057667.1, NP_570913.1, NP_001230122.1, Q9NP55-1, or Q9NP55-2. The BPIFA1 protein may have at least 75% amino acid identity to a human BPIFA1 protein, such as for example any one of NP_057667.1, NP_570913.1, NP_001230122.1, Q9NP55-1, or Q9NP55-2. The BPIFA1 protein may have at least 70% amino acid identity to a human BPIFA1 protein, such as for example any one of NP_057667.1, NP_570913.1, NP_001230122.1, Q9NP55-1, or Q9NP55-2. The BPIFA1 protein may have at least 65% amino acid identity to a human BPIFA1 protein, such as for example any one of NP_057667.1, NP_570913.1, NP_001230122.1, Q9NP55-1, or Q9NP55-2. The BPIFA1 protein may have at least 60% amino acid identity to a human BPIFA1 protein, such as for example any one of NP_057667.1, NP_570913.1, NP_001230122.1, Q9NP55-1, or Q9NP55-2.

The BPIFA1 protein may comprise at least residues 40 to 234 of a human BPIFA1 protein, such as for example any one of NP_057667.1, NP_570913.1, NP_001230122.1, Q9NP55-1, or Q9NP55-2, or a fragment thereof. The BPIFA1 protein may comprise 100% amino acid identity to residues 40 to 234 of a human BPIFA1 protein, such as for example any one of NP_057667.1, NP_570913.1, NP_001230122.1, Q9NP55-1, or Q9NP55-2. The BPIFA1 protein may comprise 95% amino acid identity to residues 40 to 234 of a human BPIFA1 protein, such as for example any one of NP_057667.1, NP_570913.1, NP_001230122.1, Q9NP55-1, or Q9NP55-2. The BPIFA1 protein may comprise 90% amino acid identity to residues 40 to 234 of a human BPIFA1 protein, such as for example any one of NP_057667.1, NP_570913.1, NP_001230122.1, Q9NP55-1, or Q9NP55-2. The BPIFA1 protein may comprise 85% amino acid identity to residues 40 to 234 of a human BPIFA1 protein, such as for example any one of NP_057667.1, NP_570913.1, NP_001230122.1, Q9NP55-1, or Q9NP55-2. The BPIFA1 protein may comprise 80% amino acid identity to residues 40 to 234 of a human BPIFA1 protein, such as for example any one of NP_057667.1, NP_570913.1, NP 001230122.1, Q9NP55-1, or Q9NP55-2. The BPIFA1 protein may comprise 75% amino acid identity to residues 40 to 234 of a human BPIFA1 protein, such as for example any one of NP_057667.1, NP_570913.1, NP_001230122.1, Q9NP55-1, or Q9NP55-2. The BPIFA1 protein may comprise 70% amino acid identity to residues 40 to 234 of a human BPIFA1 protein, such as for example any one of NP_057667.1, NP_570913.1, NP_001230122.1, Q9NP55-1, or Q9NP55-2. The BPIFA1 protein may comprise 65% amino acid identity to residues 40 to 234 of a human BPIFA1 protein, such as for example any one of NP_057667.1, NP_570913.1, NP_001230122.1, Q9NP55-1, or Q9NP55-2. The BPIFA1 protein may comprise 60% amino acid identity to residues 40 to 234 of a human BPIFA1 protein, such as for example any one of NP_057667.1, NP_570913.1, NP_001230122.1, Q9NP55-1, or Q9NP55-2.

The BPIFA1 protein may comprise at least residues 87 to 92 of a human BPIFA1 protein, such as for example any one of NP_057667.1, NP_570913.1, NP_001230122.1, Q9NP55-1, or Q9NP55-2. The BPIFA1 protein may comprise 100% amino acid identity to residues 87 to 92 of a human BPIFA1 protein, such as for example any one of NP_057667.1, NP_570913.1,

NP 001230122.1, Q9NP55-1, or Q9NP55-2. The BPIFA1 protein may comprise 95% amino acid identity to residues 87 to 92 of a human BPIFA1 protein, such as for example any one of NP_057667.1, NP_570913.1, NP_001230122.1, Q9NP55-1, or Q9NP55-2. The BPIFA1 protein may comprise 90% amino acid identity to residues 87 to 92 of a human BPIFA1 protein, such as for example any one of NP_057667.1, NP_570913.1, NP_001230122.1, Q9NP55-1, or Q9NP55-2. The BPIFA1 protein may comprise 85% amino acid identity to residues 87 to 92 of a human BPIFA1 protein, such as for example any one of NP_057667.1, NP_570913.1, NP_001230122.1, Q9NP55-1, or Q9NP55-2. The BPIFA1 protein may comprise 80% amino acid identity to residues 87 to 92 of a human BPIFA1 protein, such as for example any one of NP_057667.1, NP_570913.1, NP 001230122.1, Q9NP55-1, or Q9NP55-2. The BPIFA1 protein may comprise 75% amino acid identity to residues 87 to 92 of a human BPIFA1 protein, such as for example any one of NP_057667.1, NP_570913.1, NP_001230122.1, Q9NP55-1, or Q9NP55-2. The BPIFA1 protein may comprise 70% amino acid identity to residues 87 to 92 of a human BPIFA1 protein, such as for example any one of NP_057667.1, NP_570913.1, NP_001230122.1, Q9NP55-1, or Q9NP55-2.

### Pharmaceutical Compositions

Aspects disclosed herein comprise pharmaceutical compositions. The pharmaceutical compositions can comprise a biologically active protein and a pharmaceutically acceptable excipient and/or additive. The pharmaceutical composition can comprise two biologically active proteins. The pharmaceutical composition can comprise a biologically active protein and another biologically active compound. The composition can also contain other compatible therapeutic agents. In some embodiments, co-administration can be accomplished by co-formulation, e.g., preparing a single pharmaceutical composition including multiple compounds, e.g., the biologically active protein and another biologically active compound.

A pharmaceutical composition can comprise a biologically active protein. In some embodiments, the biologically active protein is a surfactant. In some embodiments, the surfactant is an airway surfactant. In some embodiments, the surfactant is a pulmonary surfactant. In some embodiments, the surfactant is a lung surfactant. In some embodiments, the surfactant is an alveolar surfactant. In some embodiments, the surfactant is a human surfactant. In some embodiments, the surfactant is a bovine surfactant. In some embodiments, the surfactant is a porcine surfactant. In some embodiments, the surfactant is a synthetic surfactant. In some embodiments, the surfactant is a BPI fold containing family A member 1 (BPIFA1) protein.

A pharmaceutical composition can comprise BPIFA1 protein. In some embodiments, a pharmaceutical composition comprising BPIFA1 protein comprises a therapeutically effective amount of BPIFA1 protein. A pharmaceutical composition may comprise BPIFA1 protein and a pharmaceutical excipient, e.g., a pharmaceutically acceptable excipient. A pharmaceutical composition may comprise BPIFA1 protein and a pharmaceutical additive, e.g., a pharmaceutically acceptable additive.

A pharmaceutical composition can comprise BPIFA1 protein and another biologically active compound. In some embodiments, a pharmaceutical composition comprising BPIFA1 protein may comprise a therapeutically effective amount of BPIFA1 protein. In some embodiments, a pharmaceutical composition comprising BPIFA1 protein and another biologically active compound comprises a therapeutically effective amount of BPIFA1 protein. In some embodiments, a pharmaceutical composition comprising BPIFA1 protein and another biologically active compound comprises a therapeutically effective amount of the another biologically active compound. In some embodiments, a pharmaceutical composition comprising BPIFA1 protein and the another biologically active compound comprises a therapeutically effective amount of BPIFA1 protein and a therapeutically effective amount the another biologically active compound. A pharmaceutical composition may comprise BPIFA1 protein, another biologically active compound, and a pharmaceutical excipient, e.g., a pharmaceutically acceptable excipient. A pharmaceutical composition may comprise BPIFA1 protein, another biologically active compound, and a pharmaceutical additive, e.g., a pharmaceutically acceptable additive. A pharmaceutical composition can comprise BPIFA1 protein, and another biologically active compound.

The another biologically active compound may be another protein. The another biologically active compound may be another surfactant. The another surfactant may be a pulmonary surfactant. The another surfactant may be a lung surfactant. The another surfactant may be an alveolar surfactant. The another surfactant may be an airway surfactant. The another surfactant may be derived from human. The another surfactant may be derived from bovine. The another surfactant may be derived from porcine. The another surfactant may be synthetic. Non-limiting examples of another surfactant include Curosurf®, Survanta®, Infasurf®, and Alveofact®.

The pharmaceutical composition can comprise a pharmaceutically acceptable excipient. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors and colors, and the like.

Suitable solid excipients include, but are not limited to, magnesium carbonate; magnesium stearate; talc; pectin; dextrin; starch; tragacanth; a low melting wax; cocoa butter; carbohydrates; proteins including, but not limited to, lactose, sucrose, mannitol, or sorbitol, starch from corn, wheat, rice, potato, or other plants; cellulose such as methyl cellulose, hydroxypropylmethyl-cellulose, or sodium carboxymethylcellulose; and gums including tragacanth; as well as proteins including, but not limited to, gelatin and collagen. If desired, disintegrating or solubilizing agents may be added, such as the cross-linked polyvinyl pyrrolidone, agar, alginic acid, or a salt thereof, such as sodium alginate. The pharmaceutical compositions disclosed herein can be prepared in a wide variety of oral, parenteral and topical dosage forms.

The pharmaceutical compositions can comprise a pharmaceutically acceptable carrier. Pharmaceutically acceptable carriers can be solid or liquid. Solid form preparations include powders, tablets, capsules, cachets, suppositories, and dispersible granules. A solid carrier can be one or more substances, which may also act as diluents, flavoring agents, binders, preservatives, tablet disintegrating agents, or an encapsulating material.

In powders, the carrier can be a finely divided solid, which is in a mixture with the finely divided active component. In tablets, the active component can be mixed with the carrier having the necessary binding properties in suitable proportions and compacted in the shape and size desired. The powders and tablets can contain from about 5% or about 10% to about 70% or about 80% of the compound or compounds of the present disclosure. The powders and tablets can contain from about 5% to about 90% of the compound or compounds of the present disclosure.

Liquid form preparations can include solutions, suspensions, and emulsions, for example, water or water/propylene glycol solutions. For parenteral injection, liquid preparations can be formulated in solution in aqueous polyethylene glycol solution. Formulations can be adjusted for osmolarity.

Nasal drops and oral mucosa-adhesive preparations can be prepared in powder form, aerosol form, or liquid form suitable for spray administration and the like using appropriate binders, diluents, or propellants according to conventional methods. For the preparation of the above-mentioned powder form, appropriate water-absorbing base materials may be used such as celluloses, starches, or polyacrylates and for preparation of aerosol form preparations, water, glycols, or alcohols may be used. A nonionic surfactant or the like may also be used. Formulations in the form of spray propellants can also be prepared using propellants (liquefied propellants) such as liquefied petroleum gas, carbon dioxide, fluorinated lower alkanes, and the like.

### Methods of Treatment

In one aspect, provided herein is a method of treating or preventing a respiratory infection or condition. In some embodiments, the method includes administering to a subject in need thereof a therapeutically effective amount of a biologically active protein as disclosed herein. In some embodiments, the method includes administering to a subject in need thereof a therapeutically effective amount of a surfactant as disclosed herein. The method can include administering to a subject in need thereof a therapeutically effective amount of BPIFA1 as disclosed herein.

The compounds and compositions of the present disclosure, and any other compounds, can be present in any suitable amount, and can depend on various factors including, but not limited to, weight and age of the subject, state of the disease, etc.

The method of treatment can comprise administering about 20 milligram (mg) per kilogram (kg) body weight per dose (mg/kg/dose) to about 1,000 mg/kg/dose of a biologically active airway surfactant. The method of treatment can comprise administering about 20 mg/kg/dose of a biologically active airway surfactant. The method of treatment can comprise administering about 25 mg/kg/dose of a biologically active airway surfactant. The method of treatment can comprise administering about 30 mg/kg/dose of a biologically active airway surfactant. The method of treatment can comprise administering about 35 mg/kg/dose of a biologically active airway surfactant. The method of treatment can comprise administering about 40 mg/kg/dose of a biologically active airway surfactant. The method of treatment can comprise administering about 45 mg/kg/dose of a biologically active airway surfactant. The method of treatment can comprise administering about 50 mg/kg/dose of a biologically active airway surfactant. The method of treatment can comprise administering about 54 mg/kg/dose of a biologically active airway surfactant. The method of treatment can comprise administering about 55 mg/kg/dose of a biologically active airway surfactant. The method of treatment can comprise administering about 60 mg/kg/dose of a biologically active airway surfactant. The method of treatment can comprise administering about 65 mg/kg/dose of a biologically active airway surfactant. The method of treatment can comprise administering about 70 mg/kg/dose of a biologically active airway surfactant. The method of treatment can comprise administering about 75 mg/kg/dose of a biologically active airway surfactant. The method of treatment can comprise administering about 80 mg/kg/dose of a biologically active airway surfactant. The method of treatment can comprise administering about 85 mg/kg/dose of a biologically active airway surfactant. The method of treatment can comprise administering about 90 mg/kg/dose of a biologically active airway surfactant. The method of treatment can comprise administering about 95 mg/kg/dose of a biologically active airway surfactant. The method of treatment can comprise administering about 100 mg/kg/dose of a biologically active airway surfactant. The method of treatment can comprise administering about 105 mg/kg/dose of a biologically active airway surfactant. The method of treatment can comprise administering about 108 mg/kg/dose of a biologically active airway surfactant. The method of treatment can comprise administering about 110 mg/kg/dose of a biologically active airway surfactant. The method of treatment can comprise administering about 115 mg/kg/dose of a biologically active airway surfactant. The method of treatment can comprise administering about 120 mg/kg/dose of a biologically active airway surfactant. The method of treatment can comprise administering about 125 mg/kg/dose of a biologically active airway surfactant. The method of treatment can comprise administering about 130 mg/kg/dose of a biologically active airway surfactant. The method of treatment can comprise administering about 135 mg/kg/dose of a biologically active airway surfactant. The method of treatment can comprise administering about 140 mg/kg/dose of a biologically active airway surfactant. The method of treatment can comprise administering about 145 mg/kg/dose of a biologically active airway surfactant. The method of treatment can comprise administering about 150 mg/kg/dose of a biologically active airway surfactant. The method of treatment can comprise administering about 155 mg/kg/dose of a biologically active airway surfactant. The method of treatment can comprise administering about 160 mg/kg/dose of a biologically active airway surfactant. The method of treatment can comprise administering about 165 mg/kg/dose of a biologically active airway surfactant. The method of treatment can comprise administering about 170 mg/kg/dose of a biologically active airway surfactant. The method of treatment can comprise administering about 175 mg/kg/dose of a biologically active airway surfactant. The method of treatment can comprise administering about 180 mg/kg/dose of a biologically active airway surfactant. The method of treatment can comprise administering about 185 mg/kg/dose of a biologically active airway surfactant. The method of treatment can comprise administering about 190 mg/kg/dose of a biologically active airway surfactant. The method of treatment can comprise administering about 195 mg/kg/dose of a biologically active airway surfactant. The method of treatment can comprise administering about 200 mg/kg/dose of a biologically active airway surfactant. The method of treatment can comprise administering about 205 mg/kg/dose of a biologically active airway surfactant. The method of treatment can comprise administering about 210 mg/kg/dose of a biologically active airway surfactant. The method of treatment can comprise administering about 215 mg/kg/dose of a biologically active airway surfactant. The method of treatment can comprise administering about 216 mg/kg/dose of a biologically active airway surfactant. The method of treatment can comprise administering about 220 mg/kg/dose of a biologically active airway surfactant. The method of treatment can comprise administering about 225 mg/kg/dose of a biologically active airway surfactant. The method of treatment can comprise administering about 250 mg/kg/dose of a biologically active airway surfactant. The method of treatment can comprise administering about 275 mg/kg/dose of a biologically active airway surfactant. The method of treatment can comprise administering about 300 mg/kg/dose of a biologically active airway surfactant. The method of treatment can comprise administering about 325 mg/kg/dose of a biologically active airway surfactant. The method of treatment can comprise administering about 250 mg/kg/dose of a biologically active airway surfactant. The method of treatment can comprise administering about 275 mg/kg/dose of a biologically active airway surfactant. The method of treatment can comprise administering about 400 mg/kg/dose of a biologically active airway surfactant. The method of treatment can comprise administering about 425 mg/kg/dose of a biologically active airway surfactant. The method of treatment can comprise administering about 450 mg/kg/dose of a biologically active airway surfactant. The method of treatment can comprise administering about 475 mg/kg/dose of a biologically active airway surfactant. The method of treatment can comprise administering about 500 mg/kg/dose of a biologically active airway surfactant. The method of treatment can comprise administering about 550 mg/kg/dose of a biologically active airway surfactant. The method of treatment can comprise administering about 600 mg/kg/dose of a biologically active airway surfactant. The method of treatment can comprise administering about 650 mg/kg/dose of a biologically active airway surfactant. The method of treatment can comprise administering about 700 mg/kg/dose of a biologically active airway surfactant. The method of treatment can comprise administering about 750 mg/kg/dose of a biologically active airway surfactant. The method of treatment can comprise administering about 800 mg/kg/dose of a biologically active airway surfactant. The method of treatment can comprise administering about 850 mg/kg/dose of a biologically active airway surfactant. The method of treatment can comprise administering about 900 mg/kg/dose of a biologically active airway surfactant. The method of treatment can comprise administering about 950 mg/kg/dose of a biologically active airway surfactant. The method of treatment can comprise administering about 1,000 mg/kg/dose of a biologically active airway surfactant.

The method of treatment can comprise administering about 20 milligram (mg) per kilogram (kg) body weight of the subject per dose (mg/kg/dose) to about 1,000 mg/kg/dose of BPIFA1. The method of treatment can comprise administering about 20 mg/kg/dose of BPIFA1. The method of treatment can comprise administering about 25 mg/kg/dose of BPIFA1. The method of treatment can comprise administering about 30 mg/kg/dose of BPIFA1. The method of treatment can comprise administering about 35 mg/kg/dose of BPIFA1. The method of treatment can comprise administering about 40 mg/kg/dose of BPIFA1. The method of treatment can comprise administering about 45 mg/kg/dose of BPIFA1. The method of treatment can comprise administering about 50 mg/kg/dose of BPIFA1. The method of treatment can comprise administering about 54 mg/kg/dose of BPIFA1. The method of treatment can comprise administering about 55 mg/kg/dose of BPIFA1. The method of treatment can comprise administering about 60 mg/kg/dose of BPIFA1. The method of treatment can comprise administering about 65 mg/kg/dose of BPIFA1. The method of treatment can comprise administering about 70 mg/kg/dose of BPIFA1. The method of treatment can comprise administering about 75 mg/kg/dose of BPIFA1. The method of treatment can comprise administering about 80 mg/kg/dose of BPIFA1. The method of treatment can comprise administering about 85 mg/kg/dose of BPIFA1. The method of treatment can comprise administering about 90 mg/kg/dose of BPIFA1. The method of treatment can comprise administering about 95 mg/kg/dose of BPIFA1. The method of treatment can comprise administering about 100 mg/kg/dose of BPIFA1. The method of treatment can comprise administering about 105 mg/kg/dose of BPIFA1. The method of treatment can comprise administering about 108 mg/kg/dose of BPIFA1. The method of treatment can comprise administering about 110 mg/kg/dose of BPIFA1. The method of treatment can comprise administering about 115 mg/kg/dose of BPIFA1. The method of treatment can comprise administering about 120 mg/kg/dose of BPIFA1. The method of treatment can comprise administering about 125 mg/kg/dose of BPIFA1. The method of treatment can comprise administering about 130 mg/kg/dose of BPIFA1. The method of treatment can comprise administering about 135 mg/kg/dose of BPIFA1. The method of treatment can comprise administering about 140 mg/kg/dose of BPIFA1. The method of treatment can comprise administering about 145 mg/kg/dose of BPIFA1. The method of treatment can comprise administering about 150 mg/kg/dose of BPIFA1. The method of treatment can comprise administering about 155 mg/kg/dose of BPIFA1. The method of treatment can comprise administering about 160 mg/kg/dose of BPIFA1. The method of treatment can comprise administering about 165 mg/kg/dose of BPIFA1. The method of treatment can comprise administering about 170 mg/kg/dose of BPIFA1. The method of treatment can comprise administering about 175 mg/kg/dose of BPIFA1. The method of treatment can comprise administering about 180 mg/kg/dose of BPIFA1. The method of treatment can comprise administering about 185 mg/kg/dose of BPIFA1. The method of treatment can comprise administering about 190 mg/kg/dose of BPIFA1. The method of treatment can comprise administering about 195 mg/kg/dose of BPIFA1. The method of treatment can comprise administering about 200 mg/kg/dose of BPIFA1. The method of treatment can comprise administering about 205 mg/kg/dose of BPIFA1. The method of treatment can comprise administering about 210 mg/kg/dose of BPIFA1. The method of treatment can comprise administering about 215 mg/kg/dose of BPIFA1. The method of treatment can comprise administering about 216 mg/kg/dose of BPIFA1. The method of treatment can comprise administering about 220 mg/kg/dose of BPIFA1. The method of treatment can comprise administering about 225 mg/kg/dose of BPIFA1. The method of treatment can comprise administering about 250 mg/kg/dose of BPIFA1. The method of treatment can comprise administering about 275 mg/kg/dose of BPIFA1. The method of treatment can comprise administering about 300 mg/kg/dose of BPIFA1. The method of treatment can comprise administering about 325 mg/kg/dose of BPIFA1. The method of treatment can comprise administering about 350 mg/kg/dose of BPIFA1. The method of treatment can comprise administering about 375 mg/kg/dose of BPIFA1. The method of treatment can comprise administering about 400 mg/kg/dose of BPIFA1. The method of treatment can comprise administering about 425 mg/kg/dose of BPIFA1. The method of treatment can comprise administering about 450 mg/kg/dose of BPIFA1. The method of treatment can comprise administering about 475 mg/kg/dose of BPIFA1. The method of treatment can comprise administering about 500 mg/kg/dose of BPIFA1. The method of treatment can comprise administering about 550 mg/kg/dose of BPIFA1. The method of treatment can comprise administering about 600 mg/kg/dose of BPIFA1. The method of treatment can comprise administering about 650 mg/kg/dose of BPIFA1. The method of treatment can comprise administering about 700 mg/kg/dose of BPIFA1. The method of treatment can comprise administering about 750 mg/kg/dose of BPIFA1. The method of treatment can comprise administering about 800 mg/kg/dose of BPIFA1. The method of treatment can comprise administering about 850 mg/kg/dose of BPIFA1. The method of treatment can comprise administering about 900 mg/kg/dose of BPIFA1. The method of treatment can comprise administering about 950 mg/kg/dose of BPIFA1. The method of treatment can comprise administering about 1,000 mg/kg/dose of BPIFA1.

In some embodiments, the method includes administering another compound (e.g., therapeutic compound). In some embodiments, the method includes administering another compound (e.g., therapeutic compound) in a therapeutically effective amount. In some embodiments, the another compound is another surfactant. Non-limiting examples of surfactants include lung surfactants, pulmonary surfactants, airway surfactants, and alveolar surfactants. The another surfactant may be derived from human, bovine, porcine, or synthetically. Non-limiting examples of an another surfactant include Survanta®, Infasurf®, Alveofact®, Curosurf®, or any combination thereof.

The method of treatment can comprise administering about 20 milligram (mg) per kilogram (kg) body weight per dose (mg/kg/dose) to about 1,000 mg/kg/dose of an another surfactant. The method of treatment can comprise administering about 20 mg/kg/dose of an another surfactant. The method of treatment can comprise administering about 25 mg/kg/dose of an another surfactant. The method of treatment can comprise administering about 30 mg/kg/dose of an another surfactant. The method of treatment can comprise administering about 35 mg/kg/dose of an another surfactant. The method of treatment can comprise administering about 40 mg/kg/dose of an another surfactant. The method of treatment can comprise administering about 45 mg/kg/dose of an another surfactant. The method of treatment can comprise administering about 50 mg/kg/dose of an another surfactant. The method of treatment can comprise administering about 54 mg/kg/dose of an another surfactant. The method of treatment can comprise administering about 55 mg/kg/dose of an another surfactant. The method of treatment can comprise administering about 60 mg/kg/dose of an another surfactant. The method of treatment can comprise administering about 65 mg/kg/dose of an another surfactant. The method of treatment can comprise administering about 70 mg/kg/dose of an another surfactant. The method of treatment can comprise administering about 75 mg/kg/dose of an another surfactant. The method of treatment can comprise administering about 80 mg/kg/dose of an another surfactant. The method of treatment can comprise administering about 85 mg/kg/dose of an another surfactant. The method of treatment can comprise administering about 90 mg/kg/dose of an another surfactant. The method of treatment can comprise administering about 95 mg/kg/dose of an another surfactant. The method of treatment can comprise administering about 100 mg/kg/dose of an another surfactant. The method of treatment can comprise administering about 105 mg/kg/dose of an another surfactant. The method of treatment can comprise administering about 108 mg/kg/dose of an another surfactant. The method of treatment can comprise administering about 110 mg/kg/dose of an another surfactant. The method of treatment can comprise administering about 115 mg/kg/dose of an another surfactant. The method of treatment can comprise administering about 120 mg/kg/dose of an another surfactant. The method of treatment can comprise administering about 125 mg/kg/dose of an another surfactant. The method of treatment can comprise administering about 130 mg/kg/dose of an another surfactant. The method of treatment can comprise administering about 135 mg/kg/dose of an another surfactant. The method of treatment can comprise administering about 140 mg/kg/dose of an another surfactant. The method of treatment can comprise administering about 145 mg/kg/dose of an another surfactant. The method of treatment can comprise administering about 150 mg/kg/dose of an another surfactant. The method of treatment can comprise administering about 155 mg/kg/dose of an another surfactant. The method of treatment can comprise administering about 160 mg/kg/dose of an another surfactant. The method of treatment can comprise administering about 165 mg/kg/dose of an another surfactant. The method of treatment can comprise administering about 170 mg/kg/dose of an another surfactant. The method of treatment can comprise administering about 175 mg/kg/dose of an another surfactant. The method of treatment can comprise administering about 180 mg/kg/dose of an another surfactant. The method of treatment can comprise administering about 185 mg/kg/dose of an another surfactant. The method of treatment can comprise administering about 190 mg/kg/dose of an another surfactant. The method of treatment can comprise administering about 195 mg/kg/dose of an another surfactant. The method of treatment can comprise administering about 200 mg/kg/dose of an another surfactant. The method of treatment can comprise administering about 205 mg/kg/dose of an another surfactant. The method of treatment can comprise administering about 210 mg/kg/dose of an another surfactant. The method of treatment can comprise administering about 215 mg/kg/dose of an another surfactant. The method of treatment can comprise administering about 216 mg/kg/dose of an another surfactant. The method of treatment can comprise administering about 220 mg/kg/dose of an another surfactant. The method of treatment can comprise administering about 225 mg/kg/dose of an another surfactant. The method of treatment can comprise administering about 250 mg/kg/dose of an another surfactant. The method of treatment can comprise administering about 275 mg/kg/dose of an another surfactant. The method of treatment can comprise administering about 300 mg/kg/dose of an another surfactant. The method of treatment can comprise administering about 325 mg/kg/dose of an another surfactant. The method of treatment can comprise administering about 350 mg/kg/dose of an another surfactant. The method of treatment can comprise administering about 375 mg/kg/dose of an another surfactant. The method of treatment can comprise administering about 400 mg/kg/dose of an another surfactant. The method of treatment can comprise administering about 425 mg/kg/dose of an another surfactant. The method of treatment can comprise administering about 450 mg/kg/dose of an another surfactant.

The method of treatment can comprise administering about 475 mg/kg/dose of an another surfactant. The method of treatment can comprise administering about 500 mg/kg/dose of an another surfactant. The method of treatment can comprise administering about 550 mg/kg/dose of an another surfactant. The method of treatment can comprise administering about 600 mg/kg/dose of an another surfactant. The method of treatment can comprise administering about 650 mg/kg/dose of an another surfactant. The method of treatment can comprise administering about 700 mg/kg/dose of an another surfactant. The method of treatment can comprise administering about 750 mg/kg/dose of an another surfactant. The method of treatment can comprise administering about 800 mg/kg/dose of an another surfactant. The method of treatment can comprise administering about 850 mg/kg/dose of an another surfactant. The method of treatment can comprise administering about 900 mg/kg/dose of an another surfactant. The method of treatment can comprise administering about 950 mg/kg/dose of an another surfactant. The method of treatment can comprise administering about 1,000 mg/kg/dose of an another surfactant.

The method of treatment can comprise administering about 20 milligram (mg) per kilogram (kg) body weight per day (mg/kg/day) to about 1,000 mg/kg/day of a biologically active airway surfactant. The method of treatment can comprise administering about 20 mg/kg/day of a biologically active airway surfactant. The method of treatment can comprise administering about 25 mg/kg/day of a biologically active airway surfactant. The method of treatment can comprise administering about 30 mg/kg/day of a biologically active airway surfactant. The method of treatment can comprise administering about 35 mg/kg/day of a biologically active airway surfactant. The method of treatment can comprise administering about 40 mg/kg/day of a biologically active airway surfactant. The method of treatment can comprise administering about 45 mg/kg/day of a biologically active airway surfactant. The method of treatment can comprise administering about 50 mg/kg/day of a biologically active airway surfactant. The method of treatment can comprise administering about 54 mg/kg/day of a biologically active airway surfactant. The method of treatment can comprise administering about 55 mg/kg/day of a biologically active airway surfactant. The method of treatment can comprise administering about 60 mg/kg/day of a biologically active airway surfactant. The method of treatment can comprise administering about 65 mg/kg/day of a biologically active airway surfactant. The method of treatment can comprise administering about 70 mg/kg/day of a biologically active airway surfactant. The method of treatment can comprise administering about 75 mg/kg/day of a biologically active airway surfactant. The method of treatment can comprise administering about 80 mg/kg/day of a biologically active airway surfactant. The method of treatment can comprise administering about 85 mg/kg/day of a biologically active airway surfactant. The method of treatment can comprise administering about 90 mg/kg/day of a biologically active airway surfactant. The method of treatment can comprise administering about 95 mg/kg/day of a biologically active airway surfactant. The method of treatment can comprise administering about 100 mg/kg/day of a biologically active airway surfactant. The method of treatment can comprise administering about 105 mg/kg/day of a biologically active airway surfactant. The method of treatment can comprise administering about 108 mg/kg/day of a biologically active airway surfactant. The method of treatment can comprise administering about 110 mg/kg/day of a biologically active airway surfactant. The method of treatment can comprise administering about 115 mg/kg/day of a biologically active airway surfactant. The method of treatment can comprise administering about 120 mg/kg/day of a biologically active airway surfactant. The method of treatment can comprise administering about 125 mg/kg/day of a biologically active airway surfactant. The method of treatment can comprise administering about 130 mg/kg/day of a biologically active airway surfactant. The method of treatment can comprise administering about 135 mg/kg/day of a biologically active airway surfactant. The method of treatment can comprise administering about 140 mg/kg/day of a biologically active airway surfactant. The method of treatment can comprise administering about 145 mg/kg/day of a biologically active airway surfactant. The method of treatment can comprise administering about 150 mg/kg/day of a biologically active airway surfactant. The method of treatment can comprise administering about 155 mg/kg/day of a biologically active airway surfactant. The method of treatment can comprise administering about 160 mg/kg/day of a biologically active airway surfactant. The method of treatment can comprise administering about 165 mg/kg/day of a biologically active airway surfactant. The method of treatment can comprise administering about 170 mg/kg/day of a biologically active airway surfactant. The method of treatment can comprise administering about 175 mg/kg/day of a biologically active airway surfactant. The method of treatment can comprise administering about 180 mg/kg/day of a biologically active airway surfactant. The method of treatment can comprise administering about 185 mg/kg/day of a biologically active airway surfactant. The method of treatment can comprise administering about 190 mg/kg/day of a biologically active airway surfactant. The method of treatment can comprise administering about 195 mg/kg/day of a biologically active airway surfactant. The method of treatment can comprise administering about 200 mg/kg/day of a biologically active airway surfactant. The method of treatment can comprise administering about 205 mg/kg/day of a biologically active airway surfactant. The method of treatment can comprise administering about 210 mg/kg/day of a biologically active airway surfactant. The method of treatment can comprise administering about 215 mg/kg/day of a biologically active airway surfactant. The method of treatment can comprise administering about 216 mg/kg/day of a biologically active airway surfactant. The method of treatment can comprise administering about 220 mg/kg/day of a biologically active airway surfactant. The method of treatment can comprise administering about 225 mg/kg/day of a biologically active airway surfactant. The method of treatment can comprise administering about 250 mg/kg/day of a biologically active airway surfactant. The method of treatment can comprise administering about 275 mg/kg/day of a biologically active airway surfactant. The method of treatment can comprise administering about 300 mg/kg/day of a biologically active airway surfactant. The method of treatment can comprise administering about 325 mg/kg/day of a biologically active airway surfactant. The method of treatment can comprise administering about 250 mg/kg/day of a biologically active airway surfactant. The method of treatment can comprise administering about 275 mg/kg/day of a biologically active airway surfactant. The method of treatment can comprise administering about 400 mg/kg/day of a biologically active airway surfactant. The method of treatment can comprise administering about 425 mg/kg/day of a biologically active airway surfactant. The method of treatment can comprise administering about 450 mg/kg/day of a biologically active airway surfactant. The method of treatment can comprise administering about 475 mg/kg/day of a biologically active airway surfactant. The method of treatment can comprise administering about 500 mg/kg/day of a biologically active airway surfactant. The method of treatment can comprise administering about 550 mg/kg/day of a biologically active airway surfactant. The method of treatment can comprise administering about 600 mg/kg/day of a biologically active airway surfactant. The method of treatment can comprise administering about 650 mg/kg/day of a biologically active airway surfactant. The method of treatment can comprise administering about 700 mg/kg/day of a biologically active airway surfactant. The method of treatment can comprise administering about 750 mg/kg/day of a biologically active airway surfactant. The method of treatment can comprise administering about 800 mg/kg/day of a biologically active airway surfactant. The method of treatment can comprise administering about 850 mg/kg/day of a biologically active airway surfactant. The method of treatment can comprise administering about 900 mg/kg/day of a biologically active airway surfactant. The method of treatment can comprise administering about 950 mg/kg/day of a biologically active airway surfactant. The method of treatment can comprise administering about 1,000 mg/kg/day of a biologically active airway surfactant.

The method of treatment can comprise administering about 20 milligram (mg) per kilogram (kg) body weight of the subject per day (mg/kg/day) to about 1,000 mg/kg/day of BPIFA1. The method of treatment can comprise administering about 20 mg/kg/day of BPIFA1. The method of treatment can comprise administering about 25 mg/kg/day of BPIFA1. The method of treatment can comprise administering about 30 mg/kg/day of BPIFA1. The method of treatment can comprise administering about 35 mg/kg/day of BPIFA1. The method of treatment can comprise administering about 40 mg/kg/day of BPIFA1. The method of treatment can comprise administering about 45 mg/kg/day of BPIFA1. The method of treatment can comprise administering about 50 mg/kg/day of BPIFA1. The method of treatment can comprise administering about 54 mg/kg/day of BPIFA1. The method of treatment can comprise administering about 55 mg/kg/day of BPIFA1. The method of treatment can comprise administering about 60 mg/kg/day of BPIFA1. The method of treatment can comprise administering about 65 mg/kg/day of BPIFA1. The method of treatment can comprise administering about 70 mg/kg/day of BPIFA1. The method of treatment can comprise administering about 75 mg/kg/day of BPIFA1. The method of treatment can comprise administering about 80 mg/kg/day of BPIFA1. The method of treatment can comprise administering about 85 mg/kg/day of BPIFA1. The method of treatment can comprise administering about 90 mg/kg/day of BPIFA1. The method of treatment can comprise administering about 95 mg/kg/day of BPIFA1. The method of treatment can comprise administering about 100 mg/kg/day of BPIFA1. The method of treatment can comprise administering about 105 mg/kg/day of BPIFA1. The method of treatment can comprise administering about 108 mg/kg/day of BPIFA1. The method of treatment can comprise administering about 110 mg/kg/day of BPIFA1. The method of treatment can comprise administering about 115 mg/kg/day of BPIFA1. The method of treatment can comprise administering about 120 mg/kg/day of BPIFA1. The method of treatment can comprise administering about 125 mg/kg/day of BPIFA1. The method of treatment can comprise administering about 130 mg/kg/day of BPIFA1. The method of treatment can comprise administering about 135 mg/kg/day of BPIFA1. The method of treatment can comprise administering about 140 mg/kg/day of BPIFA1. The method of treatment can comprise administering about 145 mg/kg/day of BPIFA1. The method of treatment can comprise administering about 150 mg/kg/day of BPIFA1. The method of treatment can comprise administering about 155 mg/kg/day of BPIFA1. The method of treatment can comprise administering about 160 mg/kg/day of BPIFA1. The method of treatment can comprise administering about 165 mg/kg/day of BPIFA1. The method of treatment can comprise administering about 170 mg/kg/day of BPIFA1. The method of treatment can comprise administering about 175 mg/kg/day of BPIFA1. The method of treatment can comprise administering about 180 mg/kg/day of BPIFA1. The method of treatment can comprise administering about 185 mg/kg/day of BPIFA1. The method of treatment can comprise administering about 190 mg/kg/day of BPIFA1. The method of treatment can comprise administering about 195 mg/kg/day of BPIFA1. The method of treatment can comprise administering about 200 mg/kg/day of BPIFA1. The method of treatment can comprise administering about 205 mg/kg/day of BPIFA1. The method of treatment can comprise administering about 210 mg/kg/day of BPIFA1. The method of treatment can comprise administering about 215 mg/kg/day of BPIFA1. The method of treatment can comprise administering about 216 mg/kg/day of BPIFA1. The method of treatment can comprise administering about 220 mg/kg/day of BPIFA1. The method of treatment can comprise administering about 225 mg/kg/day of BPIFA1. The method of treatment can comprise administering about 250 mg/kg/day of BPIFA1. The method of treatment can comprise administering about 275 mg/kg/day of BPIFA1. The method of treatment can comprise administering about 300 mg/kg/day of BPIFA1. The method of treatment can comprise administering about 325 mg/kg/day of BPIFA1. The method of treatment can comprise administering about 350 mg/kg/day of BPIFA1. The method of treatment can comprise administering about 375 mg/kg/day of BPIFA1. The method of treatment can comprise administering about 400 mg/kg/day of BPIFA1. The method of treatment can comprise administering about 425 mg/kg/day of BPIFA1. The method of treatment can comprise administering about 450 mg/kg/day of BPIFA1. The method of treatment can comprise administering about 475 mg/kg/day of BPIFA1. The method of treatment can comprise administering about 500 mg/kg/day of BPIFA1. The method of treatment can comprise administering about 550 mg/kg/day of BPIFA1. The method of treatment can comprise administering about 600 mg/kg/day of BPIFA1. The method of treatment can comprise administering about 650 mg/kg/day of BPIFA1. The method of treatment can comprise administering about 700 mg/kg/day of BPIFA1. The method of treatment can comprise administering about 750 mg/kg/day of BPIFA1. The method of treatment can comprise administering about 800 mg/kg/day of BPIFA1. The method of treatment can comprise administering about 850 mg/kg/day of BPIFA1. The method of treatment can comprise administering about 900 mg/kg/day of BPIFA1. The method of treatment can comprise administering about 950 mg/kg/day of BPIFA1. The method of treatment can comprise administering about 1,000 mg/kg/day of BPIFA1.

In some embodiments, the method includes administering another compound (e.g., therapeutic compound). In some embodiments, the method includes administering another compound (e.g., therapeutic compound) in a therapeutically effective amount. In some embodiments, the another compound is another surfactant. Non-limiting examples of surfactants include lung surfactants, pulmonary surfactants, airway surfactants, and alveolar surfactants. The another surfactant may be derived from human, bovine, porcine, or synthetically. Non-limiting examples of an another surfactant include Survanta®, Infasurf®, Alveofact®, Curosurf®, or any combination thereof.

The method of treatment can comprise administering about 20 milligram (mg) per kilogram (kg) body weight per day (mg/kg/day) to about 1,000 mg/kg/day of an another surfactant. The method of treatment can comprise administering about 20 mg/kg/day of an another surfactant. The method of treatment can comprise administering about 25 mg/kg/day of an another surfactant. The method of treatment can comprise administering about 30 mg/kg/day of an another surfactant. The method of treatment can comprise administering about 35 mg/kg/day of an another surfactant. The method of treatment can comprise administering about 40 mg/kg/day of an another surfactant. The method of treatment can comprise administering about 45 mg/kg/day of an another surfactant. The method of treatment can comprise administering about 50 mg/kg/day of an another surfactant. The method of treatment can comprise administering about 54 mg/kg/day of an another surfactant. The method of treatment can comprise administering about 55 mg/kg/day of an another surfactant. The method of treatment can comprise administering about 60 mg/kg/day of an another surfactant. The method of treatment can comprise administering about 65 mg/kg/day of an another surfactant. The method of treatment can comprise administering about 70 mg/kg/day of an another surfactant. The method of treatment can comprise administering about 75 mg/kg/day of an another surfactant. The method of treatment can comprise administering about 80 mg/kg/day of an another surfactant. The method of treatment can comprise administering about 85 mg/kg/day of an another surfactant. The method of treatment can comprise administering about 90 mg/kg/day of an another surfactant. The method of treatment can comprise administering about 95 mg/kg/day of an another surfactant. The method of treatment can comprise administering about 100 mg/kg/day of an another surfactant. The method of treatment can comprise administering about 105 mg/kg/day of an another surfactant. The method of treatment can comprise administering about 108 mg/kg/day of an another surfactant. The method of treatment can comprise administering about 110 mg/kg/day of an another surfactant. The method of treatment can comprise administering about 115 mg/kg/day of an another surfactant. The method of treatment can comprise administering about 120 mg/kg/day of an another surfactant. The method of treatment can comprise administering about 125 mg/kg/day of an another surfactant. The method of treatment can comprise administering about 130 mg/kg/day of an another surfactant. The method of treatment can comprise administering about 135 mg/kg/day of an another surfactant. The method of treatment can comprise administering about 140 mg/kg/day of an another surfactant. The method of treatment can comprise administering about 145 mg/kg/day of an another surfactant. The method of treatment can comprise administering about 150 mg/kg/day of an another surfactant. The method of treatment can comprise administering about 155 mg/kg/day of an another surfactant. The method of treatment can comprise administering about 160 mg/kg/day of an another surfactant. The method of treatment can comprise administering about 165 mg/kg/day of an another surfactant. The method of treatment can comprise administering about 170 mg/kg/day of an another surfactant. The method of treatment can comprise administering about 175 mg/kg/day of an another surfactant. The method of treatment can comprise administering about 180 mg/kg/day of an another surfactant. The method of treatment can comprise administering about 185 mg/kg/day of an another surfactant. The method of treatment can comprise administering about 190 mg/kg/day of an another surfactant. The method of treatment can comprise administering about 195 mg/kg/day of an another surfactant. The method of treatment can comprise administering about 200 mg/kg/day of an another surfactant. The method of treatment can comprise administering about 205 mg/kg/day of an another surfactant. The method of treatment can comprise administering about 210 mg/kg/day of an another surfactant. The method of treatment can comprise administering about 215 mg/kg/day of an another surfactant. The method of treatment can comprise administering about 216 mg/kg/day of an another surfactant. The method of treatment can comprise administering about 220 mg/kg/day of an another surfactant. The method of treatment can comprise administering about 225 mg/kg/day of an another surfactant. The method of treatment can comprise administering about 250 mg/kg/day of an another surfactant. The method of treatment can comprise administering about 275 mg/kg/day of an another surfactant. The method of treatment can comprise administering about 300 mg/kg/day of an another surfactant. The method of treatment can comprise administering about 325 mg/kg/day of an another surfactant. The method of treatment can comprise administering about 350 mg/kg/day of an another surfactant. The method of treatment can comprise administering about 375 mg/kg/day of an another surfactant. The method of treatment can comprise administering about 400 mg/kg/day of an another surfactant. The method of treatment can comprise administering about 425 mg/kg/day of an another surfactant. The method of treatment can comprise administering about 450 mg/kg/day of an another surfactant. The method of treatment can comprise administering about 475 mg/kg/day of an another surfactant. The method of treatment can comprise administering about 500 mg/kg/day of an another surfactant. The method of treatment can comprise administering about 550 mg/kg/day of an another surfactant. The method of treatment can comprise administering about 600 mg/kg/day of an another surfactant. The method of treatment can comprise administering about 650 mg/kg/day of an another surfactant. The method of treatment can comprise administering about 700 mg/kg/day of an another surfactant. The method of treatment can comprise administering about 750 mg/kg/day of an another surfactant. The method of treatment can comprise administering about 800 mg/kg/day of an another surfactant. The method of treatment can comprise administering about 850 mg/kg/day of an another surfactant. The method of treatment can comprise administering about 900 mg/kg/day of an another surfactant. The method of treatment can comprise administering about 950 mg/kg/day of an another surfactant. The method of treatment can comprise administering about 1,000 mg/kg/day of an another surfactant.

### Administration

The composition disclosed herein may be administered to treat or prevent a respiratory infection or condition. The respiratory infection or condition may be caused a respiratory virus. In some embodiments, the composition may be administered during a latent phase of a viral infection. In some embodiments, the composition may be administered during an incubation phase of a viral infection. In some embodiments, the composition may be administered during an active phase of a viral infection. In some embodiments, the composition may be administered following exposure to a respiratory virus. The exposure to a respiratory virus may be known. The exposure to a respiratory virus may be suspected. In some embodiments, the composition is administered prophylactically.

The methods described herein can comprise administering a therapeutically effective dose of a biologically active protein to a subject once. The methods described herein can comprise administering a therapeutically effective dose of a biologically active protein intratracheally to a subject once. The methods described herein can comprise administering a therapeutically effective dose of BPIFA1 to a subject once. The methods described herein can comprise administering a therapeutically effective dose of BPIFA1 intratracheally to a subject once. The methods described herein can comprise administering a therapeutically effective dose of a biologically active protein and another biologically active compound to a subject once. The methods described herein can comprise administering a therapeutically effective dose of a biologically active protein and another biologically active compound intratracheally to a subject once. The methods described herein can comprise administering a therapeutically effective dose of BPIFA1 and another biologically active compound to a subject once. The methods described herein can comprise administering a therapeutically effective dose of BPIFA1 and another biologically active compound intratracheally to a subject once. The methods described herein can comprise administering a therapeutically effective dose of BPIFA1 and Curosurf® to a subject once. The methods described herein can comprise administering a therapeutically effective dose of BPIFA1 and Curosurf® intratracheally to a subject once. The methods described herein can comprise administering a therapeutically effective dose of BPIFA1 and Survanta to a subject once. The methods described herein can comprise administering a therapeutically effective dose of BPIFA1 and Survanta intratracheally to a subject once. The methods described herein can comprise administering a therapeutically effective dose of BPIFA1 and Infasurf to a subject once. The methods described herein can comprise administering a therapeutically effective dose of BPIFA1 and Infasurf intratracheally to a subject once. The methods described herein can comprise administering a therapeutically effective dose of BPIFA1 and Alveofact to a subject once. The methods described herein can comprise administering a therapeutically effective dose of BPIFA1 and Alveofact intratracheally to a subject once.

The methods described herein can comprise administering a therapeutically effective dose of a biologically active protein to a subject twice. The methods described herein can comprise administering a therapeutically effective dose of a biologically active protein intratracheally to a subject twice. The methods described herein can comprise administering a therapeutically effective dose of BPIFA1 to a subject twice. The methods described herein can comprise administering a therapeutically effective dose of BPIFA1 intratracheally to a subject twice. The methods described herein can comprise administering a therapeutically effective dose of a biologically active protein and another biologically active compound to a subject twice. The methods described herein can comprise administering a therapeutically effective dose of a biologically active protein and another biologically active compound intratracheally to a subject twice. The methods described herein can comprise administering a therapeutically effective dose of BPIFA1 and another biologically active compound to a subject twice. The methods described herein can comprise administering a therapeutically effective dose of BPIFA1 and another biologically active compound intratracheally to a subject twice. The methods described herein can comprise administering a therapeutically effective dose of BPIFA1 and Curosurf® to a subject twice. The methods described herein can comprise administering a therapeutically effective dose of BPIFA1 and Curosurf® intratracheally to a subject twice. The methods described herein can comprise administering a therapeutically effective dose of BPIFA1 and Survanta to a subject twice. The methods described herein can comprise administering a therapeutically effective dose of BPIFA1 and Survanta intratracheally to a subject twice. The methods described herein can comprise administering a therapeutically effective dose of BPIFA1 and Infasurf to a subject twice. The methods described herein can comprise administering a therapeutically effective dose of BPIFA1 and Infasurf intratracheally to a subject twice. The methods described herein can comprise administering a therapeutically effective dose of BPIFA1 and Alveofact to a subject twice. The methods described herein can comprise administering a therapeutically effective dose of BPIFA1 and Alveofact intratracheally to a subject twice.

The methods described herein can comprise administering a therapeutically effective dose of a biologically active protein to a subject thrice. The methods described herein can comprise administering a therapeutically effective dose of a biologically active protein intratracheally to a subject thrice. The methods described herein can comprise administering a therapeutically effective dose of BPIFA1 to a subject thrice. The methods described herein can comprise administering a therapeutically effective dose of BPIFA1 intratracheally to a subject thrice. The methods described herein can comprise administering a therapeutically effective dose of a biologically active protein and another biologically active compound to a subject thrice. The methods described herein can comprise administering a therapeutically effective dose of a biologically active protein and another biologically active compound intratracheally to a subject thrice. The methods described herein can comprise administering a therapeutically effective dose of BPIFA1 and another biologically active compound to a subject thrice. The methods described herein can comprise administering a therapeutically effective dose of BPIFA1 and another biologically active compound intratracheally to a subject thrice. The methods described herein can comprise administering a therapeutically effective dose of BPIFA1 and Curosurf® to a subject thrice. The methods described herein can comprise administering a therapeutically effective dose of BPIFA1 and Curosurf® intratracheally to a subject thrice. The methods described herein can comprise administering a therapeutically effective dose of BPIFA1 and Survanta to a subject thrice. The methods described herein can comprise administering a therapeutically effective dose of BPIFA1 and Survanta intratracheally to a subject thrice. The methods described herein can comprise administering a therapeutically effective dose of BPIFA1 and Infasurf to a subject thrice. The methods described herein can comprise administering a therapeutically effective dose of BPIFA1 and Infasurf intratracheally to a subject thrice. The methods described herein can comprise administering a therapeutically effective dose of BPIFA1 and Alveofact to a subject thrice. The methods described herein can comprise administering a therapeutically effective dose of BPIFA1 and Alveofact intratracheally to a subject thrice.

The methods described herein can comprise administering a therapeutically effective dose of a biologically active protein to a subject four times. The methods described herein can comprise administering a therapeutically effective dose of a biologically active protein intratracheally to a subject four times. The methods described herein can comprise administering a therapeutically effective dose of BPIFA1 to a subject four times. The methods described herein can comprise administering a therapeutically effective dose of BPIFA1 intratracheally to a subject four times. The methods described herein can comprise administering a therapeutically effective dose of a biologically active protein and another biologically active compound to a subject four times. The methods described herein can comprise administering a therapeutically effective dose of a biologically active protein and another biologically active compound intratracheally to a subject four times. The methods described herein can comprise administering a therapeutically effective dose of BPIFA1 and another biologically active compound to a subject four times. The methods described herein can comprise administering a therapeutically effective dose of BPIFA1 and another biologically active compound intratracheally to a subject four times. The methods described herein can comprise administering a therapeutically effective dose of BPIFA1 and Curosurf® to a subject four times. The methods described herein can comprise administering a therapeutically effective dose of BPIFA1 and Curosurf® intratracheally to a subject four times. The methods described herein can comprise administering a therapeutically effective dose of BPIFA1 and Survanta to a subject four times. The methods described herein can comprise administering a therapeutically effective dose of BPIFA1 and Survanta intratracheally to a subject four times. The methods described herein can comprise administering a therapeutically effective dose of BPIFA1 and Infasurf to a subject four times. The methods described herein can comprise administering a therapeutically effective dose of BPIFA1 and Infasurf intratracheally to a subject four times. The methods described herein can comprise administering a therapeutically effective dose of BPIFA1 and Alveofact to a subject four times. The methods described herein can comprise administering a therapeutically effective dose of BPIFA1 and Alveofact intratracheally to a subject four times.

The compounds of the present disclosure can be administered at any suitable frequency, interval and duration. For example, the compound of the present disclosure may be administered only once. The compound of the present disclosure may be administered twice. The compound of the present disclosure may be administered thrice. The compound of the disclosure may be administered 4 times. Any two administrations may be administered 2 hours apart, 4 hours apart, 6hours apart, 8 hours apart, 10 hours apart, 12 hours apart, 14 hours apart, 16 hours apart, 18 hours apart, 20 hours apart, 22 hours apart, or 24 hours apart. The dosage of the first administration of the compounds disclosed herein may be higher than subsequent administrations of the compounds disclosed herein. The dosage of the first and second administration of the compounds disclosed herein may be higher than subsequent administrations of the compounds disclosed herein. The dosage for all administrations of the compounds disclosed herein may differ from one another. The dosage for all administrations of the compounds disclosed herein may be the same as one another. Any two administrations may, for example, be administered an hour, or two, three or more hours apart, a day, or two, three, or more days apart, or 2, 3, 4, 5, 6, or 7 days apart, so as to provide the preferred dosage level. The compound of the present disclosure can be administered once an hour, or two, three or more times an hour, once a day, or two, three, or more times per day, or once every 2, 3, 4, 5, 6, or 7 days, so as to provide the preferred dosage level. When the compound of the present disclosure is administered more than once a day, representative intervals include 5, 10, 15, 20, 30, 45 and 60 minutes, as well as 1, 2, 4, 6, 8, 10, 12, 16, 20, and 24 hours. The compound of the present disclosure can be administered once, twice, thrice or four or more times. The compound of the present disclosure can be administered once, twice, thrice or four or more times, for an hour, for 1 to 6 hours, for 1 to 12 hours, for 1 to 24 hours, for 6 to 12 hours, for 12 to 24 hours, for a single day, for 1 to 7 days, for a single week, for 1 to 4 weeks, for a month, for 1 to 12 months, for a year or more, or even indefinitely.

The compounds described herein can be used in combination with one another, or with adjunctive agents that may not be effective alone but may contribute to the efficacy of the active agent. The compounds of the present disclosure can be co-administered with another active agent. Co-administration includes administering the compound of the present disclosure and active agent within 0.5, 1, 2, 4, 6, 8, 10, 12, 16, 20, or 24 hours of each other. Co-administration also includes administering the compound of the present disclosure and active agent simultaneously, approximately simultaneously (e.g., within about 1, 5, 10, 15, 20, or 30 minutes of each other), or sequentially in any order. Moreover, the compound of the present disclosure and the active agent can each be administered once a day, or two, three, or more times per day so as to provide the preferred dosage level per day.

The compound of the present disclosure and the active agent can be present in the compositions of the present disclosure in any suitable weight ratio, such as from about 1:100 to about 100:1 (w/w), or about 1:50 to about 50:1, or about 1:25 to about 25:1, or about 1:10 to about 10:1, or about 1:5 to about 5:1 (w/w). The compound of the present disclosure and the other active agent can be present in any suitable weight ratio, such as about 1:100 (w/w), 1:50, 1:25, 1:10, 1:5, 1:4, 1:3, 1:2, 1:1, 2:1, 3:1, 4:1, 5:1, 10:1, 25:1, 50:1 or 100:1 (w/w). Other dosages and dosage ratios of the compound of the present disclosure and the active agent are suitable in the compositions and methods of the present disclosure.

The compounds and compositions of the present disclosure can be delivered by any suitable means, including intratracheal. The pharmaceutical preparation is preferably in unit dosage form. In such form the preparation is subdivided into unit doses containing appropriate quantities of the compounds and compositions of the present disclosure. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The pharmaceutical compositions disclosed herein can be administered intratracheally. Also, the pharmaceutical compositions described herein can be administered by inhalation, for example, intranasally or orally.

The compositions disclosed herein can be administered by inhalation. The compositions disclosed herein can be administered by an incubation tube. The compositions disclosed herein can be administered as an aerosol. The compositions disclosed herein can be administered by infusion. The compositions disclosed herein can be administered by bolus.

### Diseases, Conditions, and Disorders

In some embodiments, the compositions and methods of the present disclosure are useful for treating a subject with a respiratory infection or condition. In some embodiments, the compositions and methods of the present disclosure are useful prophylactically to prevent a subject from developing a respiratory infection or condition. In some embodiments, the compositions and methods of the present disclosure are useful prophylactically to prevent a subject from developing complications from a respiratory infection or condition. Non-limiting examples of respiratory infections or conditions include bronchiolitis, common cold, croup, influenza, influenza-like illness, pneumonia, severe acute respiratory syndrome (SARS), Middle East respiratory syndrome (MERS), and coronavirus disease (COVID-19). In some embodiments, the respiratory infection or condition is caused by a respiratory virus. Non-limiting examples of respiratory viruses include adenoviruses, coronaviruses, parainfluenza viruses, parvoviruses, respiratory syncytial viruses (RSV), rhinoviruses, enteroviruses, metapneumoviruses (MPV), e.g., Human Metapneumovirus (HMPV)), and influenza viruses.

### Subjects

In some aspects, the subject is a mammal. In some aspects, the subject is a human being. In some aspects, the subject is a human patient. In some aspects, the subject may be at risk of having a respiratory infection or condition. The subject may be at risk of developing a respiratory infection or condition. The subject may have been exposed to a bacteria or virus that causes a respiratory infection or condition. The subject may be at risk for exposure to a bacteria or virus that causes a respiratory infection or condition. In some aspects, the subject can have or is suspected of having an infection or condition such as a respiratory infection or condition. In some aspects, the subject is healthy. The subject may have a respiratory infection or condition. The subject may be suspected of having a respiratory infection or condition. The subject may have or be suspected of having a respiratory infection or condition as disclosed herein. The subject may be susceptible to a respiratory infection or condition. The subject may be at risk of contracting a respiratory infection or condition. The subject may be at risk of complications from a respiratory infection or condition. The subject may have a preexisting condition. Non-limiting examples of preexisting conditions include a heart condition, lung condition, neuromuscular condition, and immune condition. As an example, a heart condition may be a congenital heart condition. As another example, a lung condition may be a chronic lung condition and/or cystic fibrosis. As yet another example, an immune condition may be a condition rendering the subject immunocompromised. The preexisting condition may be bronchopulmonary dysplasia.

The subject can be male. The subject can be female. The subject can be intersex. The gender of the subject can be determined according to the definitions of the American Medical Association (AMA). The subject can be a neonate, infant, child, adolescent, adult or senior according to the definitions of the American Medical Association (AMA). The subject can be a pediatric subject (e.g., a neonate, an infant, a child, or an adolescent) or an adult subject (e.g., a young adult, a middle-aged adult, or a senior adult)). In some embodiments, the subject is an infant, e.g., a human infant. In some embodiments, the infant is a premature infant (e.g., a premature human infant). In some embodiments, the premature infant (e.g., premature human infant) has a gestational age of less than 37, 36, 35, 34, 33, 32, 31, 30, 29, 28, or 27 weeks. In some embodiments, the subject is a child. In some embodiments, the subject is an adolescent. In some embodiments, the subject is an adult. In some embodiments, the subject is less than 20 years of age, less than 19 years of age, less than 18 years of age, less than 17 years of age, less than 16 years of age, less than 15 years of age, or less than 14 years of age, less than 13 years of age, less than 12 years of age, less than 11 years of age, less than 10 years of age, less than 9 years of age, less than 8 years of age, less than 7 years of age, less than 6 years of age, less than 5 years of age, less than 4 years of age, less than 3 years of age, less than 2 years of age, or less than 1 year of age. In some embodiments, the subject is less than 14 years of age. In some embodiments, the subject is less than 13 years of age. In some embodiments, the subject is less than 12 years of age. In some embodiments, the subject is less than 11 years of age. In some embodiments, the subject is less than 10 years of age. In some embodiments, the subject is less than 9 years of age. In some embodiments, the subject is less than 8 years of age. In some embodiments, the subject is less than 7 years of age. In some embodiments, the subject is less than 6 years of age. In some embodiments, the subject is less than 5 years of age. In some embodiments, the subject is less than 4 years of age. In some embodiments, the subject is less than 3 years of age. In some embodiments, the subject is less than 2 years of age. In some embodiments, the subject is less than 1 year of age. In some embodiments, the subject is less than 12 months of age. In some embodiments, the subject is less than 11 months of age. In some embodiments, the subject is less than 10 months of age. In some embodiments, the subject is less than 9 months of age. In some embodiments, the subject is less than 8 months of age. In some embodiments, the subject is less than 7 months of age. In some embodiments, the subject is less than 6 months of age. In some embodiments, the subject is less than 5 months of age. In some embodiments, the subject is less than 4 months of age. In some embodiments, the subject is less than 3 months of age. In some embodiments, the subject is less than 2 months of age. In some embodiments, the subject is less than 1 month of age. In some embodiments, the subject is less than 4 weeks of age. In some embodiments, the subject is less than 3 weeks of age. In some embodiments, the subject is less than 2 weeks of age. In some embodiments, the subject is less than 1 weeks of age. In some embodiments, the subject is less than 14 days of age. In some embodiments, the subject is less than 13 days of age. In some embodiments, the subject is less than 12 days of age. In some embodiments, the subject is less than 11 days of age. In some embodiments, the subject is less than 10 days of age. In some embodiments, the subject is less than 9 days of age. In some embodiments, the subject is less than 8 days of age. In some embodiments, the subject is less than 7 days of age. In some embodiments, the subject is less than 6 days of age. In some embodiments, the subject is less than 5 days of age. In some embodiments, the subject is less than 4 days of age. In some embodiments, the subject is less than 3 days of age. In some embodiments, the subject is less than 2 days of age. In some embodiments, the subject is less than 1 day of age. In some embodiments, the subject is a senior. In some embodiments, the subject is greater than about 50 years of age. In some embodiments, the subject is greater than about 55 years of age. In some embodiments, the subject is greater than about 60 years of age. In some embodiments, the subject is greater than about 65 years of age. In some embodiments, the subject is greater than about 70 years of age. In some embodiments, the subject is greater than about 75 years of age. In some embodiments, the subject is greater than about 80 years of age. In some embodiments, the subject is greater than about 85 years of age. In some embodiments, the subject is greater than about 70 years of age. In some embodiments, the subject is greater than about 75 years of age. In some embodiments, the subject is greater than about 80 years of age. In some embodiments, the subject is greater than about 85 years of age. In some embodiments, the subject is greater than about 90 years of age. In some embodiments, the subject is greater than about 95 years of age. In some embodiments, the subject is greater than about 100 years of age.

### Additional Enumerated Embodiments

Embodiment 1. A method for preventing or treating a respiratory infection in a human subject comprising: administering to the human subject a therapeutically effective amount of an airway surfactant protein, wherein the respiratory infection is caused by a respiratory virus.

Embodiment 2. The method of embodiment 1, wherein the respiratory virus comprises adenovirus, coronavirus, parainfluenza, parvovirus, respiratory syncytial virus (RSV), or any combination thereof.

Embodiment 3. The method of any one of the preceding embodiments, wherein the respiratory virus is RSV.

Embodiment 4. The method of any one of the preceding embodiments, wherein the airway surfactant is administered during the latent phase.

Embodiment 5. The method of any one of the preceding embodiments, wherein the airway surfactant is administered during the incubation phase.

Embodiment 6. The method of any one of the preceding embodiments, wherein the airway surfactant is administered following known exposure to a respiratory virus.

Embodiment 7. The method of any one of the preceding embodiments, wherein the airway surfactant is administered prophylactically.

Embodiment 8. The method of any one of the preceding embodiments, wherein the airway surfactant is administered following suspected exposure to a respiratory virus.

Embodiment 9. The method of any one of the preceding embodiments, wherein the subject is susceptible to the respiratory infection.

Embodiment 10. The method of any one of the preceding embodiments, wherein the subject is at risk of contracting the respiratory infection.

Embodiment 11. The method of any one of the preceding embodiments, wherein the subject is at risk for complications from the respiratory infection.

Embodiment 12. The method of any one of the preceding embodiments, wherein the human subject is male, female or intersex according to American Medical Associations' definitions.

Embodiment 13. The method of embodiment 12, wherein the human subject is a neonate, infant, child, adolescent, adult or senior according to American Medical Associations' definitions.

Embodiment 14. The method of embodiment 13, wherein the human subject is a neonate, infant, or child according to American Medical Associations' definitions.

Embodiment 15. The method of embodiment 14, wherein the infant is premature.

Embodiment 16. The method of embodiment 13, wherein the human subject is a senior according to American Medical Associations' definitions.

Embodiment 17. The method of embodiment 16, wherein the human has a preexisting condition.

Embodiment 18. The method of embodiment 17, wherein the preexisting condition comprises a heart, lung, neuromuscular condition, immune condition, or any combination thereof.

Embodiment 19. The method of embodiment 18, wherein the preexisting condition comprises a lung, neuromuscular condition, immune condition, or any combination thereof.

Embodiment 20. The method of embodiment 18, wherein the heart condition comprises a congenital heart condition.

Embodiment 21. The method of embodiment 18, wherein the lung condition comprises a chronic lung condition.

Embodiment 22. The method of embodiment 18, wherein the immune condition comprises being immunocompromised.

Embodiment 23. The method of embodiment 18, wherein the lung condition comprises cystic fibrosis.

Embodiment 24. The method of embodiment 1, wherein the airway surfactant comprises a human airway surfactant.

Embodiment 25. The method of embodiment 24, wherein the human airway surfactant comprises human BPIFA1protein.

Embodiment 26. The method of embodiment 25, wherein the human airway surfactant has 90% amino acid identity to human BPIFA1 protein.

Embodiment 27. The method of embodiment 25, wherein the human airway surfactant has 80% amino acid identity to human BPIFA1 protein.

Embodiment 28. The method of embodiment 25, wherein the human airway surfactant has 70% amino acid identity to human BPIFA1 protein.

Embodiment 29. The method of embodiment 25, wherein the human airway surfactant comprises residues 40 to 234 of human BPIFA1 protein.

Embodiment 30. The method of embodiment 29, wherein the human airway surfactant comprises 90% amino acid identity residues 40 to 234 of human BPIFA1 protein.

Embodiment 31. The method of embodiment 29, wherein the human airway surfactant comprises 80% amino acid identity residues 40 to 234 of human BPIFA1 protein.

Embodiment 32. The method of embodiment 29, wherein the human airway surfactant comprises 70% amino acid identity residues 40 to 234 of human BPIFA1 protein.

Embodiment 33. The method of embodiment 25, wherein the human airway surfactant comprises at least residues 87-92 of human BPIFA1 protein.

Embodiment 34. The method of embodiment 1, wherein the airway surfactant is administered by inhalation.

Embodiment 35. The method of embodiment 1, wherein the airway surfactant is administered through an incubation tube.

Embodiment 36. The method of embodiment 1, wherein the airway surfactant is administered as an aerosol.

Embodiment 37. The method of embodiment 1, wherein the airway surfactant is administered by an infusion.

Embodiment 38. The method of embodiment 1, wherein the airway surfactant is administered by a bolus.

Embodiment 39. The method of embodiment 1, wherein the airway surfactant is administered as a single dose.

Embodiment 40. The method of embodiment 1, wherein the airway surfactant is administered as several doses.

Embodiment 41. The method of embodiment 40, wherein the several doses comprise two doses.

Embodiment 42. The method of embodiment 41, wherein the two doses are administered 24 hours apart.

Embodiment 43. The method of embodiment 41, wherein the two doses are administered 12 hours apart.

Embodiment 44. The method of embodiment 41, wherein the two doses are administered 8 hours apart.

Embodiment 45. The method of embodiment 41, wherein the two doses are administered 6 hours apart.

Embodiment 46. The method of embodiment 41, wherein the two doses are administered 4 hours apart.

Embodiment 47. The method of embodiment 1, further comprising administering the airway surfactant in combination with another surfactant.

Embodiment 48. The method of embodiment 47, wherein the another surfactant is an airway surfactant or an alveolar surfactant.

Embodiment 49. The method of embodiment 47, wherein the another surfactant is from porcine, bovine, or synthetic origin.

Embodiment 50. The method of embodiment 47, wherein the another surfactant is selected from any one of the following Survanta®, Infasurf®, Alveofact®, and Curosurf®.

Embodiment 51. The method of embodiment 47, wherein the another surfactant is Curosurf®.

Embodiment 52. A method for preventing or treating a respiratory infection in a subject comprising: administering to the subject a therapeutically effective amount of an airway surfactant.

Embodiment 53. The method of embodiment 52, wherein the airway surfactant comprises a therapeutically active portion of BPIFA1.

### EXAMPLES

### Example 1—RSV Infection of Stem Cell Derived Airway Epithelial Cells to Test BPIFA1 as Prophylactic RSV Antiviral

Cells for RSV infection were prepared in plates by coating plates with 100 μL of 0.1 mg/ml of Matrigel and left overnight prior to plating cells. iPSC derived airway organoids dissociated and plated onto Matrigel coated 96-well imaging plate at 20,000 cells per well. Cells expanded in Pneumacult™ Ex+ media for 3 days, or until well reached 90-100% confluency.

Cells were treated with either 100 μM of recombinant BPIFA1 or 1 mg/mL Curosurf® (Poractant alfa). Recombinant BPIFA1 or recombinant BPIFA1 and Curosurf® was mixed with RSV and allowed to incubate in Eppendorf tubes at room temperature for 10-15 minutes. Pneumacult™Ex+ media was removed from airway epithelial cells in the 96-well plate. Fifty μL DMEM/F12 was added to 12 wells as the RSV negative control. Forty μL DMEM/F12 and 10 μL RSV (4,540 infectious units (ifu)/mL) was added to 16 wells as the RSV positive control. Forty μL DMEM/F12 and 10 μL RSV (4,540 infectious units (ifu)/mL) plus 100 μM BPIFA1 was added to 16 wells. Forty μL DMEM/F12 and 10 μL RSV (4,540 infectious units (ifu)/mL) plus 1 mg/mL Curosurf® was added to 16 wells. The solutions were incubated with the cells for 2 hours and then removed. The infection rate was analyzed 96 hours post-infection.

Following the 96-hours post-infection, cells in 96-well imaging plate were fixed with 4% PFA for 30 mins. RSV infection rate was assessed by immunofluorescent staining for an RSV nucleoprotein. Staining volume for each well of the 96-well plate was 50 μL. Cells were incubated with an RSV nucleoprotein primary antibody used at 1:100 dilution at 4° C. overnight. The antibody was washed 3 times in PBS prior to a secondary antibody stain. The secondary antibody (fluorescent secondary antibody, Donkey Anti-Mouse Alexa Fluor® 488 used at 1:400) was incubated with cells at room temperature, in the dark, for 1 hour. The secondary antibody was then washed 3 times in PBS. Nuclear stain was performed using 10 μg/mL Hoechst for 3 minutes. No observable cell death was detected by assessing by confluency of wells (brightfield imaging) as well as number of DAPI positive cells per well.

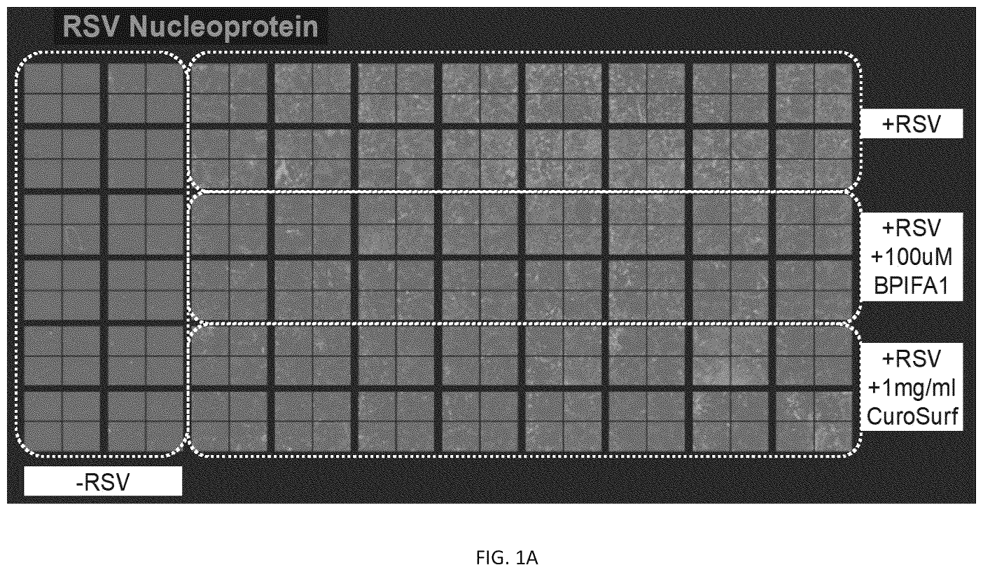

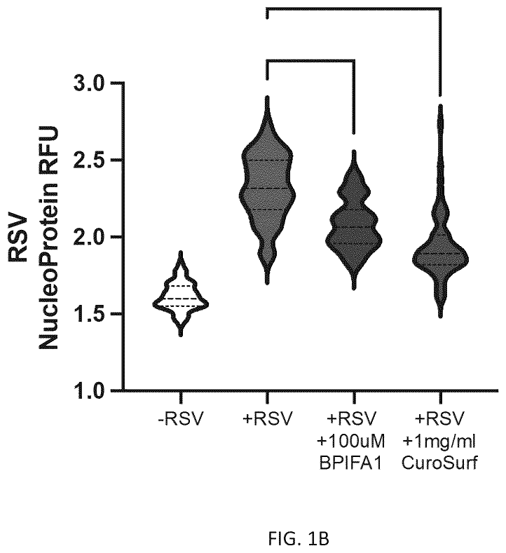

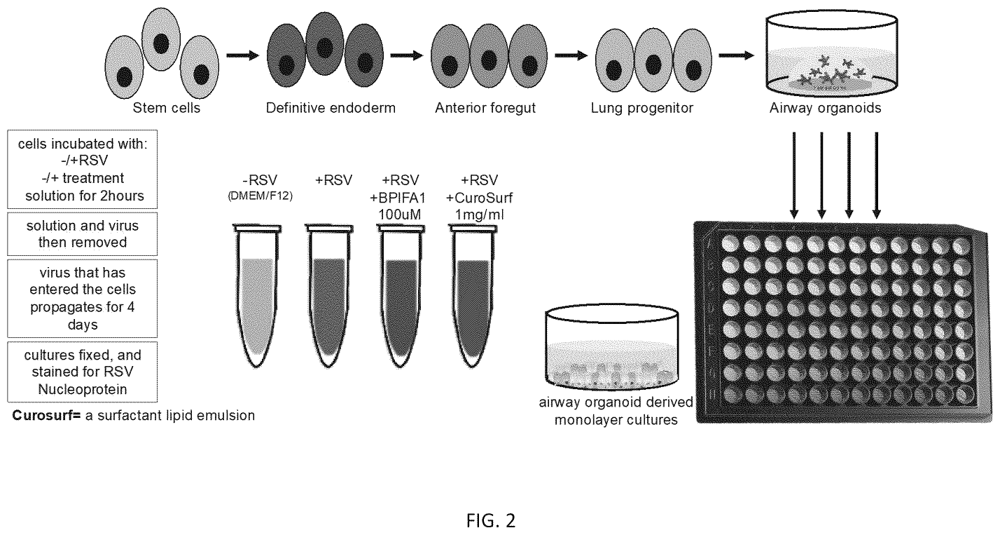

Plates were imaged at 10x magnification with 4 images taken per well. Infection was assessed based on fluorescent intensity (488 channel) of each well (FIG. 1A). Relative fluorescence of RSV nucleoprotein (measure for infection) statistics were performed by t-test and graphed (FIG. 1B). Wells on the periphery of the plate were excluded from treatment and analysis to avoid variable edge effect. The workflow described herein is presented in FIG. 2.

Recombinant BPIFA1 effectively reduces RSV infection when applied to airway epithelial cells. BPIFA1 mixed with RSV which was then applied to cells, had subsequently lower infection rates compared to the non-treated control.

### Example 2—Addition of Exogenous Surfactant BPIFA1 and Curosurf® Reduces RSV Infection

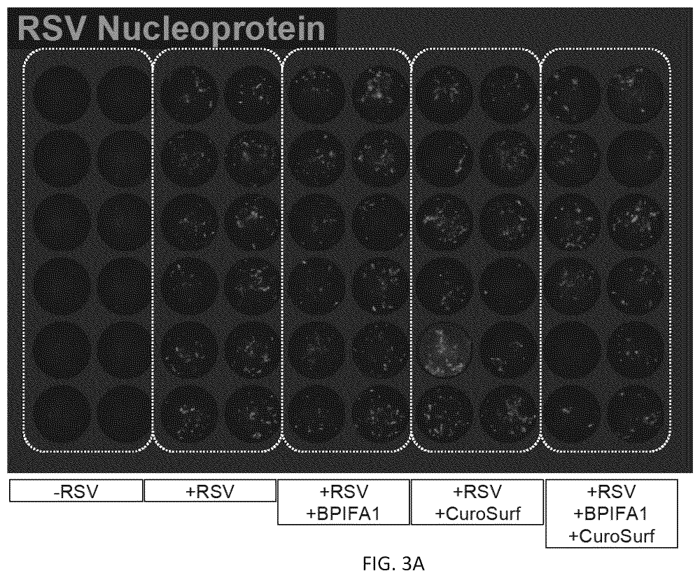

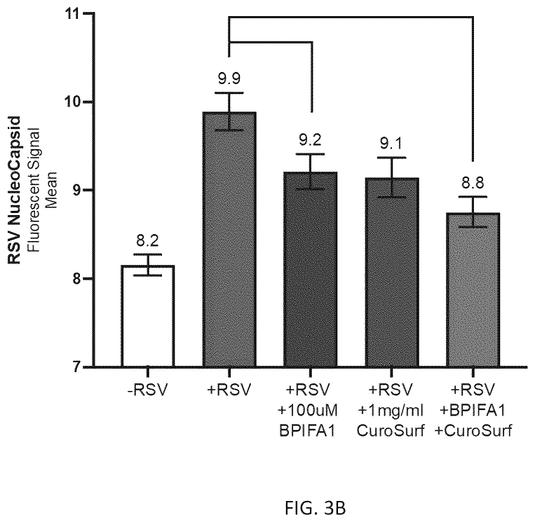

Airway surfactant protein, BPIFA1 was assessed for as a Respiratory Syncytial Virus (RSV) antiviral. Airway epithelial cells were produced as described in Example 1 and were pre-treated with 100 μM BPIFA1, 1 mg/mL Curosurf®, or 100 μM BPIFA1 and 1 mg/mL Curosurf®. The prophylactic surfactant treatments were applied to the airway cultures 1 hour prior to RSV exposure. Then, RSV was added to the wells, incubated for 2 hours before the RSV +/−surfactant treatments was removed, and infection allowed to progress over 4 days. Immunofluorescent staining for RSV nucleoprotein was performed and imaged via plate scan on the Incucyte as described in Example 1. A whole well scan at 4x magnification was performed for the 12 biologic replicates per condition (FIG. 3A). The combination of BPIFA1 and Curosurf® had the greatest antiviral abilities (p-value=0.0001) (FIG. 3B).

Recombinant BPIFA1 effectively reduces RSV infection when applied to airway epithelial cells. Cells treated with BPIFA1 one hour prior to introduction of RSV to the culture, had lower infection rates compared to the non-treated control. Additionally, the combination of BPIFA1 and Curosurf® had a synergistic RSV antiviral effect.

### Example 3—Prophylactic Use of One Dose of BPIFA1 on a Premature Infant

An infant is born premature and is intubated in the neonatal intensive care unit (NICU). There are many cases of respiratory syncytial virus (RSV) in the hospital placing the infant at risk of a serious infection from RSV. The infant weighs 2.5 kg and is administered a first dose of 500 mg recombinant BPIFA1 as a liquid surfactant emulsion through an intubation tube. The first dose may be given all at one or broken up into two aliquots.

### Example 4—Prophylactic Use of Multiple Doses of BPIFA1 on a Premature Infant

Continuing with Example 3, the risk for RSV continues to remain high and although not required the hospital staff opts to administer a second dose of 250 mg recombinant BPIFA1 as a liquid surfactant emulsion through an intubation tube to the infant 12 hours after receiving their first dose of recombinant BPIFA1. If the hospital staff is still concerned about RSV infection, a third dose of 250 mg recombinant BPIFA1 as a liquid surfactant emulsion through an intubation tube to the infant may be administered 12 hours after receiving their second dose of recombinant BPIFA1.

### Example 5—Prophylactic Use of One Dose of BPIFA1 on a Patient

An intubated patient is being cared for in the intensive care unit (ICU) in a hospital. There are many cases of respiratory syncytial virus (RSV) in the hospital placing the patient at risk of a serious infection from RSV. The patient weighs 48 kg and is administered a first dose of 4,800 mg recombinant BPIFA1. The first dose may be broken up into multiple aliquots and administered to the patient over an hour. Similar to Example 4, the patient may be given follow up doses. The patient in the ICU may be a senior. The patient in the ICU may have a preexisting condition.

### Example 6—Prophylactic Use of BPIFA1 on a Senior Patient

A senior living in a nursing home with a high number of RSV cases is at risk for a serious infection from RSV given their age. The nursing home doctor gives the senior an inhalant comprising recombinant BPIFA1 and instructs the senior to take 2 puffs on the inhaler every 12 hours for a total of 3 days. The senior weighs 48 kg. Each puff on the inhaler comprises 500 mg of recombinant BPIFA1. After the 3 days the senior has been administered 6,000 mg of recombinant BPIFA1.

### Example 7—Use of BPIFA1 on a Patient Presenting Symptoms of RSV Infection

A senior frequents a senior center that has recently had several people associated with the center identified as RSV cases. They begin to exhibit mild cold-like symptoms, similar to many of their friends at the senior center that ended up having RSV. The senior goes to the doctor and is given an inhalant comprising recombinant BPIFA1 and is instructed to take 3 puffs on the inhaler every 12 hours for a total of 3 days. Following the first 3 days, the senior is instructed to take 1 puff of the inhaler 3 times a day (morning, afternoon, and night) for 7 days. The senior weighs 48 kg. Each puff on the inhaler comprises 500 mg of recombinant BPIFA1. After the 3 days the senior has been administered 9,000 mg of recombinant BPIFA1. After the full 10 days the senior has been administered a total of 19,500 mg recombinant BPIFA1.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1. A method for preventing or treating a respiratory infection in a human subject comprising: administering to the human subject a therapeutically effective amount of a BPI fold-containing family A member 1 (BPIFA1) protein, wherein the respiratory infection is caused by a respiratory virus.

2. The method of claim 1, wherein the respiratory virus comprises adenovirus, coronavirus, parainfluenza, parvovirus, respiratory syncytial virus (RSV), or any combination thereof.

3. The method of claim 2, wherein the respiratory virus is RSV.

4.-6. (canceled)

7. The method of claim 1, wherein the BPIFA1 protein is administered prophylactically.

8.-11. (canceled)

12. The method of claim 1, wherein the human subject is male, female or intersex according to American Medical Association definitions.

13. The method of claim 12, wherein the human subject is a neonate, infant, child, adolescent, adult or senior according to American Medical Association definitions.

14. The method of claim 13, wherein the human subject is a neonate, infant, or child according to American Medical Association definitions.

15. The method of claim 14, wherein the infant is premature.

16. The method of claim 13, wherein the human subject is a senior according to American Medical Association definitions.

17.-23. (canceled)

24. The method of claim 1, wherein the BPIFA1 protein comprises a human BPIFA1 protein.

25. The method of claim 24, wherein the BPIFA1 protein has at least 80% amino acid identity to the human BPIFA1 protein.

26. The method of claim 25, wherein the BPIFA1 protein has at least 90% amino acid identity to the human BPIFA1 protein.

27.-28. (canceled)

29. The method of claim 25, wherein the BPIFA1 protein comprises residues 40 to 234 of the human BPIFA1 protein.

30.-32. (canceled)

33. The method of claim 25, wherein the BPIFA1 protein comprises at least residues 87-92 of the human BPIFA1 protein.

34. The method of claim 1, wherein the BPIFA1 protein is administered by inhalation.

35.-46. (canceled)

47. The method of claim 1, further comprising administering the BPIFA1 protein in combination with a surfactant.

48.-49. (canceled)

50. The method of claim 47, wherein the surfactant is selected from the group consisting of Survanta®, Infasurf®, Alveofact®, and Curosurf®.

51. The method of claim 47, wherein the surfactant is Curosurf®.

52. The method of claim 1, wherein the therapeutically effective amount of the BPIFA1 protein per dose is 25 mg/kg/dose, 50 mg/kg/dose, 100 mg/kg/dose, 150 mg/kg/dose, 200 mg/kg/dose, 250 mg/kg/dose, 300 mg/kg/dose, 350 mg/kg/dose, 400 mg/kg/dose, 450 mg/kg/dose, or 500 mg/kg/dose or an amount of BPIFA1 protein per dose within a range defined by any of the preceding values.

53. The method of claim 1, wherein the therapeutically effective amount of the BPIFA1 protein is 25 mg/kg/day, 50 mg/kg/day, 100 mg/kg/day, 150 mg/kg/day, 200 mg/kg/day, 250 mg/kg/day, 300 mg/kg/day, 350 mg/kg/day, 400 mg/kg/day, 450 mg/kg/day, 500 mg/kg/day, or 1000 mg/kg/day, or an amount of BPIFA1 protein within a range defined by any of the preceding values.
